(19) United States
(12) Patent Application Publication
(10) Pub. No.: US 2025/0302296 A1
(43) Pub. Date: Oct. 2, 2025

(54) VISUAL FIELD MEASUREMENT METHOD AND MOVING BODY

(71) Applicant: HONDA MOTOR CO., LTD., Tokyo (JP)

(72) Inventors: Misako Koike, Wako-shi (JP); Kosuke Nakanishi, Wako-shi (JP); Gakuyo Fujimoto, Wako-shi (JP)

(73) Assignee: HONDA MOTOR CO., LTD., Tokyo (JP)

(21) Appl. No.: 19/039,944

(22) Filed: Jan. 29, 2025

(30) Foreign Application Priority Data
Mar. 28, 2024 (JP) 2024-054471

(51) Int. Cl. A61B 3/024

(52) U.S. Cl. CPC A61B 3/024; A61B 2503/22

(57) ABSTRACT

A method for determining a visual field of a subject is provided. The method includes relatively moving a target with respect to the subject, and determining a visual field of the subject based on a position of the target at a point in time when the subject has directed a line of sight of the subject to the target relatively moving with respect to the subject.

### CROSS-REFERENCE TO RELATED APPLICATION(S)

This application claims priority to and the benefit of Japanese Patent Application No. 2024-054471, filed Mar. 28, 2024, the entire disclosure of which is incorporated herein by reference.

### BACKGROUND OF THE INVENTION

### Field of the Invention

The present invention relates to a visual field measurement method and a moving body.

### Description of the Related Art

There is a known technique that uses the visual field of a driver to provide driving assistance. Japanese Patent Laid-Open No. 2023-119428 describes that a visual field inspection is performed by irradiating a windshield with light.

### SUMMARY OF THE INVENTION

The inventors have found that the visual field for a moving target can be different from the visual field for a stationary target. Some aspects of the present invention provide a technique for measuring a visual field according to a situation.

According to some embodiments, a method for determining a visual field of a subject, the method comprising: relatively moving a target with respect to the subject; and determining a visual field of the subject based on a position of the target at a point in time when the subject has directed a line of sight of the subject to the target relatively moving with respect to the subject is provided.

### BRIEF DESCRIPTION OF THE DRAWINGS

FIG. 1 is a schematic diagram for describing a configuration example of a vehicle according to some embodiments;

FIG. 2 is a flowchart for describing an example of a visual field measurement method according to some embodiments;

FIG. 3 is a schematic diagram for describing an example of a test case according to some embodiments;

FIGS. 4A to 4D are schematic diagrams for describing an example of a test case according to some embodiments;

FIGS. 5A and 5B are schematic diagrams for describing an example of a visual field according to some embodiments;

FIG. 6 is a diagram for describing an example of visual field information according to some embodiments;

FIG. 7 is a flowchart for describing an example of a driving assistance method according to some embodiments; and

FIG. 8 is a schematic diagram for describing an example of a driving assistance situation according to some embodiments.

### DESCRIPTION OF THE EMBODIMENTS

Hereinafter, embodiments will be described in detail with reference to the attached drawings. Note, the following embodiments are not intended to limit the scope of the claimed invention, and limitation is not made to an invention that requires a combination of all features described in the embodiments. Two or more of the multiple features described in the embodiments may be combined as appropriate. Furthermore, the same reference numerals are given to the same or similar configurations, and redundant description thereof is omitted.

### <Control Device and Application Example Thereof>

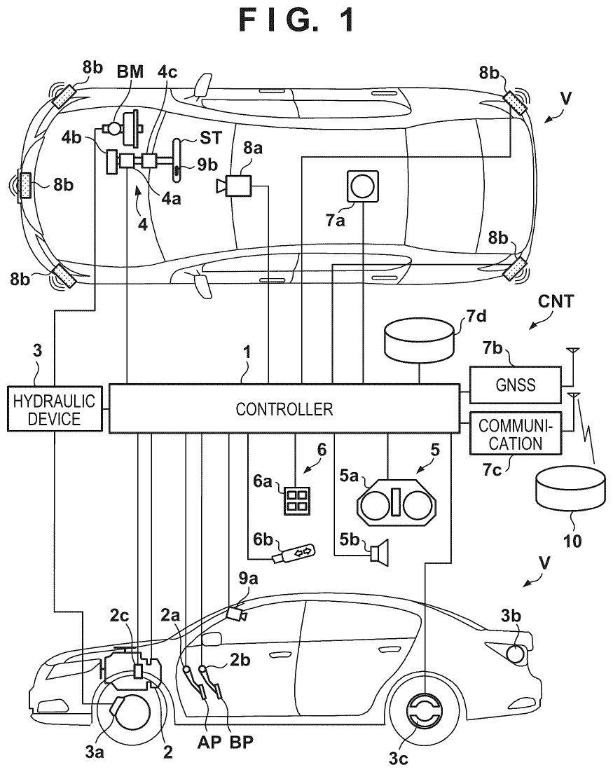

FIG. 1 is a block diagram of a control device CNT according to some embodiments, and is also a schematic diagram of a vehicle V, which is an application example thereof. In FIG. 1, an outline of the vehicle V is shown in a plan view and a side view. The vehicle V in the present embodiment is, as an example, a sedan-type four-wheeled passenger vehicle, and can be, for example, a parallel hybrid vehicle. The vehicle Vis not limited to the four-wheeled passenger vehicle, and may be a straddle type vehicle (a two-wheeled or three-wheeled motorcycle) or a large-sized vehicle such as a truck or a bus.

The control device CNT includes a controller 1, which is an electronic circuit that performs control of the vehicle V, including driving assistance of the vehicle V. The controller 1 includes a plurality of electronic control units (ECUs). For example, an ECU is provided for each function of the control device CNT. Each ECU includes a processor represented by a central processing unit (CPU), a storage device such as a semiconductor memory, an interface with an external device, and the like. The storage device stores a program to be executed by the processor, data used for processing by the processor, and the like. The interface includes an input and output interface, and a communication interface. Each ECU may include a plurality of processors, a plurality of storage devices, and a plurality of interfaces. A program to be stored in the storage device may be installed in the control device CNT using a storage medium such as a CD-ROM so as to be stored in the storage device. Additionally or alternatively, the program to be stored in the storage device may be downloaded from an external server via wireless communication.

The controller 1 controls drive (acceleration) of the vehicle V by controlling a power unit (power plant) 2. The power unit 2 is a travelling drive unit that outputs a driving force for rotating driving wheels of the vehicle V, and can include an internal combustion engine, a motor, and an automatic transmission. The motor can be used as a drive source for accelerating the vehicle V, and can also be used as a generator at the time of deceleration or the like (regenerative braking).

In the present embodiment, the controller 1 controls outputs of the internal combustion engine and the motor, or switches a gear ratio of the automatic transmission in correspondence with a driver's drive operation detected by an operation detection sensor 2a provided in an accelerator pedal AP and an operation detection sensor 2b provided in a brake pedal BP, a vehicle speed of the vehicle V detected by a rotation speed sensor 2c, and the like. The automatic transmission is provided with the rotation speed sensor 2c that detects the rotation speed of an output shaft of the automatic transmission as a sensor that detects a traveling state of the vehicle V. It is possible to calculate the vehicle speed of the vehicle V from a detection result of the rotation speed sensor 2c.

The controller 1 controls braking (deceleration) of the vehicle V by controlling a hydraulic device 3. A driver's braking operation on the brake pedal BP is converted into hydraulic pressure in a brake master cylinder BM and transmitted to the hydraulic device 3. The hydraulic device 3 is an actuator capable of controlling a hydraulic pressure of a hydraulic oil supplied to a brake device 3a (for example, a disc brake device) provided on each of the four wheels based on the hydraulic pressure transmitted from the brake master cylinder BM.

The controller 1 can control braking of the vehicle V by performing drive control of an electromagnetic valve or the like included in the hydraulic device 3. The controller 1 can also configure an electric servo brake system by controlling the distribution of the braking force by the brake device 3a and the braking force by the regenerative braking of the motor included in the power unit 2. The controller 1 may turn on a brake lamp 3b at the time of braking.

The controller 1 controls steering of the vehicle V by controlling an electric power steering device 4. The electric power steering device 4 includes a mechanism for steering front wheels in response to a driver's drive operation (steering operation) on a steering wheel ST. The electric power steering device 4 includes a drive unit 4a that exerts a driving force (may be noted as steering assist torque) for assist in the steering operation or automatic steering of the front wheels of the vehicle V). The drive unit 4a includes a motor as a drive source. In addition, the electric power steering device 4 further includes a steering angle sensor 4b that detects a steering angle, and a torque sensor 4c that detects steering torque (also, referred to as steering load torque, and is distinguished from steering assist torque) borne by a driver.

The controller 1 controls electric parking brake devices 3c provided in respective rear wheels of the vehicle V. The electric parking brake device 3c includes a mechanism for locking the rear wheels. The controller 1 is capable of controlling locking and unlocking of the rear wheels by the electric parking brake device 3c.

The controller 1 controls an information output device 5 that notifies the inside of the vehicle of information. The information output device 5 includes, for example, a display device 5a that notifies the driver of information by images, and/or a sound output device 5b that notifies the driver of information by sound. Examples of the display device 5a include a display device provided in an instrument panel, and a display device provided in the steering wheel ST. In addition, the display device 5a may include a head-up display. The information output device 5 may notify an occupant of information by vibration or light.

The controller 1 receives an instruction input from the occupant (for example, the driver) via an input device 6. The input device 6 is disposed at a position operable by the driver, and includes, for example, a switch group 6a that is used when the driver gives an instruction for the vehicle V, and/or a blinker lever 6b for operating a direction indicator (blinker).

The controller 1 recognizes and determines a current position and a course (an attitude) of the vehicle V. In the present embodiment, the vehicle V includes a gyro sensor 7a, a global navigation satellite system (GNSS) sensor 7b, and a communication device 7c. The gyro sensor 7a detects a rotational motion (yaw rate) of the vehicle V. The GNSS sensor 7b detects a current position of the vehicle V. In addition, the communication device 7c performs wireless communication with a server that provides map information and traffic information, and acquires these pieces of information. Furthermore, the communication device 7c may read visual field information from a database 10. The visual field information is information to be used to estimate the visual field of the driver of the vehicle V. Details of the visual field information will be described later.

The controller 1 determines the course of the vehicle V, based on detection results of the gyro sensor 7a and the GNSS sensor 7b, also sequentially acquires map information about the course from the server via the communication device 7c, and stores the map information in a database 7d (a storage device). The vehicle V may also include another sensor for detecting a state of the vehicle V, such as an acceleration sensor for detecting acceleration of the vehicle V.

The controller 1 assists the driving of the vehicle V, based on detection results of various detection units provided in the vehicle V. The vehicle V includes a plurality of surrounding detection units 8a and 8b serving as an external sensor that detects the outside (surrounding situation) of the vehicle V, and a plurality of in-vehicle detection units 9a and 9b serving as an in-vehicle sensor that detects a state inside the vehicle (the state of occupants, particularly, the driver). The controller 1 can grasp the situation surrounding the vehicle V based on the detection results of the surrounding detection units 8a and 8b and then assist the driving of the vehicle V in correspondence with this surrounding situation. In addition, the controller 1 can determine whether the driver is performing a predetermined operation obligation imposed on the driver when assisting the driving based on the detection results of the in-vehicle detection units 9a to 9b.

The surrounding detection unit 8a is an imaging device (hereinafter, it may be referred to as a front camera 8a) that captures an image of the front of the vehicle V, and is attached to the vehicle interior side of the windshield at the front of the roof of the vehicle V, for example. The controller 1 can extract a contour of a target or a lane marking (such as a white line) on a road by analyzing an image captured by the front camera 8a.

The surrounding detection unit 8b is a millimeter wave radar (hereinafter, it may be referred to as a radar 8b), detects a target around the vehicle V using radio waves, and detects (measures) a distance to the target and a direction (azimuth) of the target with respect to the vehicle V. In the example shown in FIG. 1, five radars 8b are provided, one at the center of the front portion of the vehicle V, one at each of the left and right corner portions of the front portion, and one at each of the left and right corner portions of the rear portion.

The surrounding detection units provided in the vehicle V are not limited to the above configuration. The number of cameras and the number of radars may be changed. A light detection and ranging (LiDAR) for detecting a target around the vehicle V may be provided.

The in-vehicle detection unit 9a is an imaging device (hereinafter, it may be referred to as an in-vehicle camera 9a) that captures an image of the inside of the vehicle, and is attached to the vehicle interior side at the front of the roof of the vehicle V, for example. In the present embodiment, the in-vehicle camera 9a is a driver monitor camera that captures an image of the driver (for example, driver's eyes and face). The controller 1 can determine the direction of the line of sight and the face of the driver by analyzing an image (a face image of the driver) captured by the in-vehicle camera 9a.

The in-vehicle detection unit 9b is a grip sensor (hereinafter, referred it may be referred to as a grip sensor 9b) that detects the driver gripping the steering wheel ST, and is provided on, for example, at least a part of the steering wheel ST. As the in-vehicle detection units, a torque sensor 4c that detects the steering torque of the driver may be used.

### <Visual Field Measurement Method>

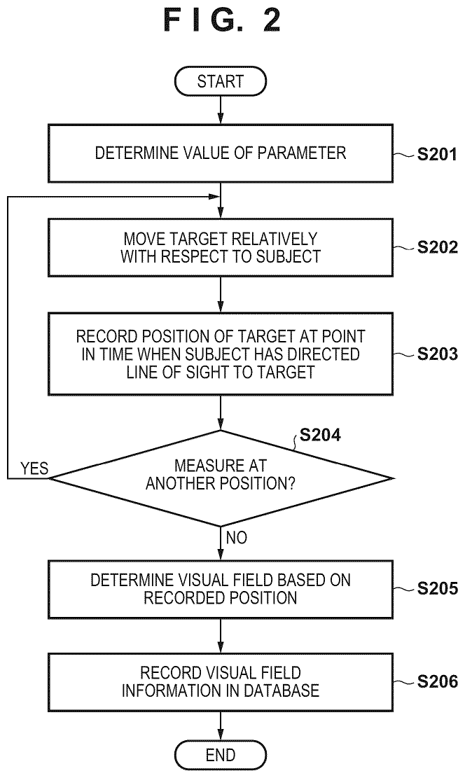

A method for measuring a visual field of a person according to some embodiments will be described with reference to FIG. 2. In the following description, a person whose visual field is to be measured is referred to as a subject, and a person who measures a visual field is referred to as a measurer. The method of FIG. 2 may be performed for each of a plurality of subjects. The visual field of one subject may be measured by a plurality of measurers.

In the method of FIG. 2, the visual field of a subject is measured in one test case. To start the method of FIG. 2, a measurer guides the subject to a fixed position of a measurement place. During the measurement of the visual field, the subject may remain at the same position without moving from the fixed position. Alternatively, the subject may continue to move during the measurement of the visual field. In this specification, when it is described that an object moves without specifying a criterion, it means that the object moves with respect to the ground. During the measurement of the visual field, the subject may stand upright, sit on a chair or the like, or walk. Alternatively, the visual field of the subject may be measured while the subject is on a moving body such as the vehicle V. As a result, the visual field of the subject is measured in a situation similar to driving a moving body. The moving body may be any moving body such as an automobile, a bicycle, or an electric wheelchair. During the measurement of the visual field, the subject may move the vehicle V or may keep the vehicle V stationary. The driving of the vehicle V during the measurement of the visual field may be performed by automated driving or by the measurer.

In S201, the measurer determines a value of a parameter to be used in a test case to be performed. The value of the parameter may be preset for each test case. The parameter may include at least one of (1) a movement characteristic of a test target, (2) a movement characteristic of a subject, (3) a direction of a line of sight of the subject, (4) a color of the test target, (5) a light environment in which a visual field is to be measured, (6) an index indicating the number of traffic participants to be arranged around the test target, and (7) a shape of the test target. Any of these parameters can affect the visual field of the subject. Each parameter will be described below.

### (1) Movement Characteristic of Test Target

The test target is a target that is relatively moved with respect to the subject in order to determine the boundary of the visual field of the subject. The test target may be a traffic participant. The traffic participant may include a pedestrian, a bicycle driver, a vehicle, or the like. The test target may be a flying object such as a drone. The test target may be a target other than the traffic participant.

The visual field of the subject may vary depending on the movement characteristic of the test target. Therefore, the test case may specify the movement characteristic of the test target. The test target moves with the movement characteristic specified in the test case during the measurement of the visual field. The movement characteristic may be defined by at least one of a speed, acceleration, a movement direction, a movement route, straightness of movement, and rhythm of movement. The speed may be, for example, 0 km/h (that is, stationary), 10 km/h, 20 km/h, 30 km/h, or the like. The acceleration may be, for example, 0G (that is, constant speed), 0.5G, 1G (for example, natural fall), or the like. The movement direction may be defined by, for example, an angle formed by the direction of the line of sight of the subject and the movement direction of the test target. The movement route may be defined by, for example, a curvature. The straightness of movement may define, for example, movement in a straight line or meandering movement. The rhythm of movement may define continuous movement with a constant rhythm or movement with intermittent pauses.

### (2) Movement Characteristic of Subject

The visual field of the subject may vary depending on a movement characteristic of the subject. Therefore, the test case may specify the movement characteristic of the subject. The subject moves with the movement characteristic specified in the test case during the measurement of the visual field. Examples of the movement characteristic of the subject may be the same as the examples of the movement characteristic of the test target.

### (3) Direction of Line of Sight of Subject

The visual field of the subject may vary depending on the direction of the line of sight of the subject during the measurement. Therefore, the test case may specify the direction of the line of sight of the subject during the measurement. The direction of the line of sight of the subject may be defined by a combination of an elevation/depression angle and an azimuth angle with respect to the direction in front of the trunk of the subject. For example, the test case may specify directing the line of sight in the direction in front of the trunk of the subject, directing the line of sight in the direction at an angle of 45 degrees to the right in front of the trunk of the subject, or directing the line of sight in the direction at an angle of 45 degrees to the upper in front of the trunk of the subject.

### (4) Color of Test Target

The visual field of the subject may vary depending on the color of the test target. Therefore, the test case may specify the color of the test target. The color of the test target may be selected from a plurality of colors (for example, black, red, blue, yellow, and the like). If the test target is not a single color, a color that occupies a majority of the appearance of the test target may be regarded as the color of the test target.

### (5) Light Environment

The visual field of the subject may vary depending on the light environment of the measurement place. Therefore, the test case may specify the light environment. The light environment may be an environment related to a light amount, a position of a light source, a color (wavelength) of light, and the like. For example, examples of light environment can include daytime, nighttime, backlighting, front lighting, specific weather (sunny, cloudy), and the like. The measurer may illuminate the measurement place with a light or the like to adjust the light environment.

### (6) Index Indicating the Number of Traffic Participants to be Arranged Around Test Target

The visual field of the subject may vary depending on the number of traffic participants included in the visual field of the subject. Therefore, the test case may specify the index indicating the number of traffic participants to be arranged around the test target. The index indicating the number of traffic participants may be the number of traffic participants itself or a category of the number of traffic participants (for example, four categories of 0 people, 1 to 5 people, 6 to 10 people, and 11 or more people).

### (7) Shape of Test Target

The visual field of the subject may vary depending on the shape of the test target. Therefore, the test case may specify the shape of the test target. The shape of the test target may be, for example, a human (pedestrian), a bicycle driver, a vehicle, or the like.

In S202, the measurer relatively moves the test target with respect to the subject. Specifically, the measurer may move the test target with the color and movement characteristic determined in S201, may move the subject with the movement characteristic determined in S201, or may perform both. This movement may be performed in the light environment determined in S201. The measurer may arrange one or more traffic participants of the index determined in S201 around the target object.

Before starting the movement, the measurer instructs the subject to direct the line of sight in the direction determined in S201. Furthermore, before starting the movement, the measurer instructs the subject to look at the test target that is about to move out of the visual field, and to look at the test target that has moved into the visual field from outside the visual field.

In step S203, the measurer records the position of the test target at a point in time when the subject has directed the line of sight to the test target relatively moving with respect to the subject. The measurer may determine the point in time when the subject has directed the line of sight to the test target based on a report from the subject. Alternatively or additionally, in a case where the subject is on the vehicle V, the direction of the line of sight of the subject may be detected using the in-vehicle camera 9a of the vehicle V. The measurer may determine the point in time when the subject has directed the line of sight to the test target based on a change in the line of sight detected by the in-vehicle camera 9a. The position of the test target may be defined by a combination of an elevation/depression angle and an azimuth angle in the direction of the test target with respect to the direction in front of the trunk of the measurer and a distance from the trunk of the measurer to the test target. The position of the test target may be the position of any one point of the test target, for example, the center of the test target.

In S204, the measurer determines whether the measurement is performed by arranging the test target at another position with respect to the subject. When it is determined that the measurement is performed by arranging the test target at another position (“YES” in S204), the measurer repeats the steps of S202 to S203. When it is determined that the measurement is not performed by arranging the test target at another position (“NO” in S204), the measurer performs S205. In this manner, the measurer records various positions with respect to the subject in S203.

In S205, the measurer determines the visual field of the subject based on the record in S205 performed one or more times. Specifically, the measurer determines, as the visual field, a three-dimensional region whose outer edge is the position recorded in S205 performed one or more times.

In S206, the measurer records visual field information in the database 10. The visual field information includes the value of the parameter used to measure the visual field and the visual field determined in S205. The parameter may include at least one of (1) the movement characteristic of the test target, (2) the movement characteristic of the subject, (3) the direction of the line of sight of the subject, (4) the color of the test target, (5) the light environment in which the visual field has been measured, (6) the index indicating the number of traffic participants arranged around the test target, and (7) the shape of the test target. The direction of the line of sight of the subject can align with the direction of the line of sight of the subject before the subject directs the line of sight to the test target.

The visual field of one subject may be measured in a plurality of test cases. In that case, the method of FIG. 2 may be performed for each of the test cases. The test cases may specify different values of parameters. For example, the measurer may determine, for each of a plurality of directions with respect to the subject, the visual field of the subject when the test target is moved while the line of sight of the subject is directed in an individual direction. The measurer may determine, for each of a plurality of test targets with different shapes, the visual field of the subject when an individual test target is relatively moved with respect to the subject. The measurer may determine, for each of a plurality of movement characteristics, the visual field of the subject when the test target is moved with an individual movement characteristic. The measurer may determine, for each of a plurality of movement characteristics, the visual field of the subject when the subject is moved with an individual movement characteristic. The measurer may determine, for each of a plurality of light environments, the visual field of the subject in an individual light environment. The measurer may determine, for each of a plurality of test targets with different colors, the visual field of the subject when an individual test target is relatively moved with respect to the subject. The measurer may determine, for each of a plurality of indexes indicating the number of traffic participants, the visual field of the subject when traffic participants of an individual index are arranged.

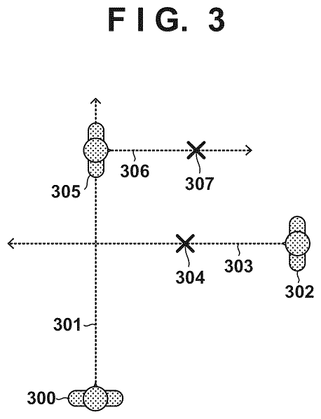

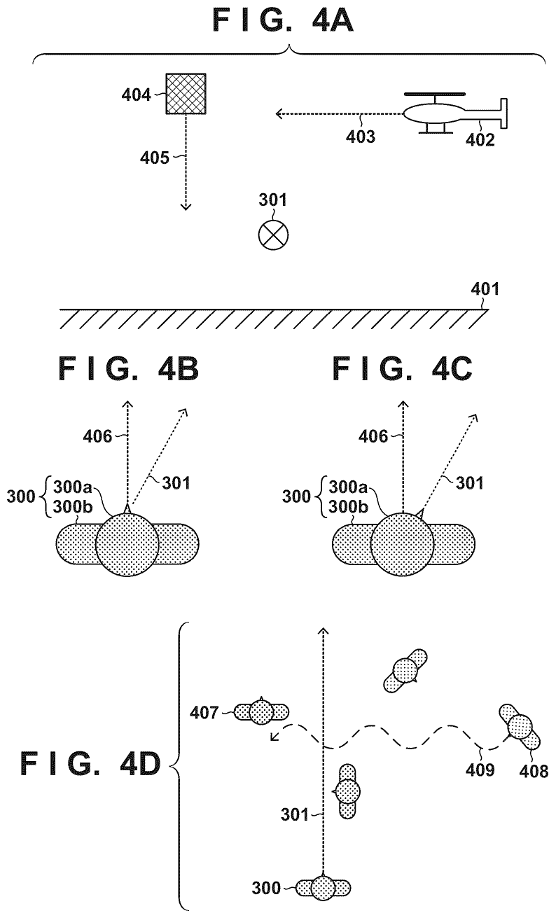

An example of the test case will be described with reference to FIGS. 3 to 4D. FIGS. 3 and 4B to 4D show a state in which the measurement place is viewed from above a subject 300. FIG. 4A shows the visual field of the subject 300. In the example of FIG. 3, the subject 300 is instructed to direct the line of sight in a direction 301 during the measurement of the visual field. That is, the direction 301 represents the direction of the line of sight of the subject 300 during the measurement of the visual field.

The measurer moves a test target 302 along a route 303 so as to approach the direction 301. In addition, the measurer also moves a test target 305 along a route 306 so as to be away from the direction 301. It is assumed that the subject 300 looks at the test target 302 at a point in time when the test target 302 reaches a position 304. In this case, the measurer records the position 304 with respect to the subject 300 at this point in time. Similarly, it is assumed that subject 300 looks at the test target 305 at a point in time when the test target 305 reaches a position 307. In this case, the measurer records the position 307 with respect to the subject 300 at this point in time.

In the example of FIG. 3, the two test targets 302 and 305 move in one execution of S202 to S203. Alternatively, in one execution of S202 to S203, only one test target may move, or three or more test targets may move. In addition, the route may be set at an arbitrary angle with respect to the direction 301.

In the example of FIG. 3, the test targets move on the ground. Alternatively, as shown in FIG. 4A, a test target 402 may move along a route 403 in which the test target 402 flies over a ground surface 401. In addition, a test target 404 may move along a route 405 in which the test target 404 falls toward the ground surface 401.

In the test cases shown in FIGS. 4B and 4C, the measurer instructs the subject 300 to face in the direction 301 deviated from a direction 406 in front of a trunk 300b of the subject 300. As shown in FIG. 4B, the measurer may instruct the subject 300 to direct a face 300a in the direction 406 in front of the trunk 300b. Alternatively, as shown in FIG. 4C, the measurer may instruct the subject 300 to direct the face 300a in the direction 301.

In the test case shown in FIG. 4D, three pedestrians 407 are arranged around a test target 408 as traffic participants. The pedestrians 407 may walk freely or remain at the same positions during the measurement of the visual field. Traffic participants other than pedestrians may be arranged, or traffic participants may not be arranged as shown in FIG. 4D. The test target 408 moves along a meandering route 409.

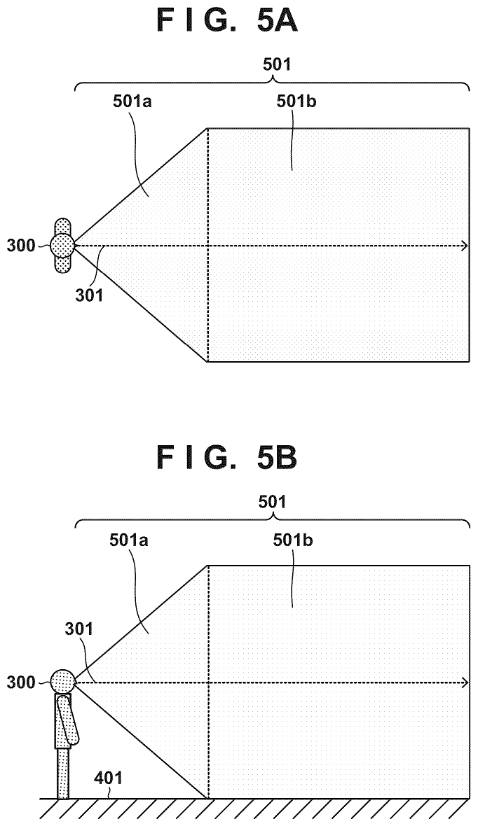

FIGS. 5A and 5B show an example of a visual field obtained by experiments by the inventors. FIG. 5A shows a visual field 501 of the subject 300 as viewed from above. FIG. 5B shows the visual field 501 of the subject 300 as viewed from the side. The visual field 501 has a shape formed by a conical portion 501a having the subject 300 as a vertex, and a cylindrical portion 501b extending from a bottom surface of the conical portion 501a.

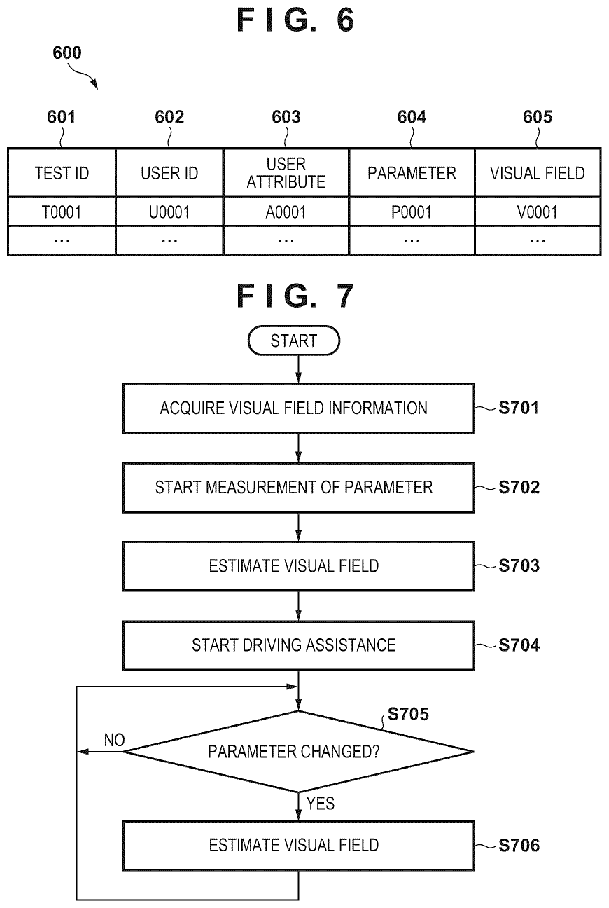

FIG. 6 shows an example of visual field information 600 recorded in the database 10 in S206 of FIG. 2. In FIG. 6, the visual field information 600 is recorded in a table format, but the visual field information 600 may be recorded in another format. The visual field information 600 has one entry for each execution of the method of FIG. 2. In the visual field information 600, information in columns 601 to 605 is recorded in association with each other. The column 601 stores identification information for identifying one test. The column 602 stores identification information for identifying a subject. The column 603 stores a value of an attribute of the subject. The measurer may record values of attributes that may affect the visual field as part of the visual field information 600. Such attributes may include, for example, at least one of an age category, an eyesight category, with or without glasses, with or without contact lenses, and with or without a specific disease (for example, glaucoma). The age category may be divided into 10-year increments or other granularity. The eyesight category may be divided into 0.1 increments or other granularity. The column 604 stores the value of the parameter used to measure the visual field. The column 605 stores the visual field determined in S205.

An administrator of the database 10 may integrate a plurality of entries in the visual field information 600 to generate a new entry. For example, the administrator may integrate a plurality of entries with the same attribute value (column 603) and the same parameter value (column 604) to generate a new entry. The columns 603 and 604 for the new entry store the information before the integration. The column 605 for the new entry stores a visual field representing the visual fields of the plurality of entries before the integration (for example, the union, intersection, or the like). The newly generated entry may be used as generic visual field information on a specific attribute value. Furthermore, the administrator may integrate a plurality of entries with the same the parameter values (column 604) to generate a new entry. The column 604 for the new entry stores the information before the integration. The column 605 for the new entry stores a visual field representing the visual fields of the plurality of entries before the integration (for example, the union, intersection, or the like). The newly generated entry may be used as general-purpose visual field information independent of the attributes.

### <Driving Assistance Method>

A driving assistance method to be performed by the vehicle V will be described with reference to FIG. 7. Each step of FIG. 7 may be performed by the control device CNT. Specifically, each step of FIG. 7 may be performed by a processor of the control device CNT executing a program that has been read into a memory of the control device CNT. Alternatively, some or all of the functional steps of FIG. 7 may be realized by a dedicated integrated circuit such as an application specific integrated circuit (ASIC). The method of FIG. 7 may be started in response to an instruction to start driving assistance from a driver of the vehicle V (hereinafter, simply referred to as a driver), or may be automatically started in response to the power of the vehicle V being turned on. The method of FIG. 7 may be performed by another moving body (for example, an airplane, a ship, or the like) other than the vehicle V.

In S701, the control device CNT acquires the visual field information 600 from the database 10. When the control device CNT can identify the driver and the visual field information 600 about this driver is recorded in the database 10, the control device CNT may acquire the visual field information 600 about this driver. In other cases, the control device CNT may acquire the general-purpose visual field information 600.

In S702, the control device CNT starts measurement of a parameter for estimating the visual field of the driver of the vehicle V. Thereafter, the control device CNT continues to measure the parameter. The parameter may include at least one of (1) a movement characteristic of a target around the vehicle V, (2) a movement characteristic of the driver, (3) a direction of the line of sight of the driver, (4) a color of the target around the vehicle V, (5) a light environment around the vehicle V, (6) an index indicating the number of traffic participants to be arranged around the vehicle V, and (7) a shape of the target around the vehicle V.

The movement characteristic of the target around the vehicle Vis measured based on, for example, detection results of the front camera 8a and the radar 8b. The movement characteristic of the driver is measured based on detection results of the rotation speed sensor 2c, the gyro sensor 7a, and the like. The direction of the line of sight of the driver is measured based on, for example, a detection result of the in-vehicle camera 9a. The color of the target around the vehicle V is measured based on, for example, a detection result of the front camera 8a. The light environment around vehicle V is measured based on, for example, a detection result of the front camera 8a. The index indicating the number of traffic participants around the vehicle Vis measured based on, for example, detection results of the front camera 8a and the radar 8b. The shape of the target around the vehicle Vis measured based on, for example, detection results of the front camera 8a and the radar 8b.

In S703, the control device CNT estimates the visual field associated with the value of the parameter measured in S702 in the visual field information 600 as the visual field of the driver. If there is no visual field information 600 with a value matching the value of the parameter measured in S702, the control device CNT may determine the visual field information 600 with a value closest to the value of the parameter measured in S702. The visual field included in the visual field information 600 has the shape of the visual field 501 described in FIGS. 5A and 5B. Therefore, the control device CNT estimates the visual field of the driver so as to have a shape formed by a conical portion having the driver as a vertex and a cylindrical portion extending from the bottom surface of the conical portion.

In S704, the control device CNT provides driving assistance based on the visual field estimated in S703. Driving assistance may include notifying the driver of the presence of traffic participants. For example, when there is a possibility that a target positioned outside the visual field of the driver (for example, a traffic participant or a falling object) collides with the vehicle V, the control device CNT may notify the driver of the presence of the target. Alternatively or additionally, when the line of sight of the driver is not directed to a traffic participant although the traffic participant has been in the visual field of the driver for a predetermined time or more, the control device CNT may notify the driver of the presence of a traffic participant.

In S705, the control device CNT determines whether the value of the parameter for which measurement has been started in S702 has changed. When it is determined that the value of the parameter has changed (“YES” in S705), the control device CNT shifts the processing to S706, and in the other case (“NO” in S705), the processing repeats S705. In this manner, the control device CNT waits until the value of the parameter changes.

In S706, the control device CNT estimates the visual field associated with the changed value of the parameter as the visual field of the driver, similarly to S703. In this manner, the control device CNT updates the visual field to be used for driving assistance each time the value of the parameter changes.

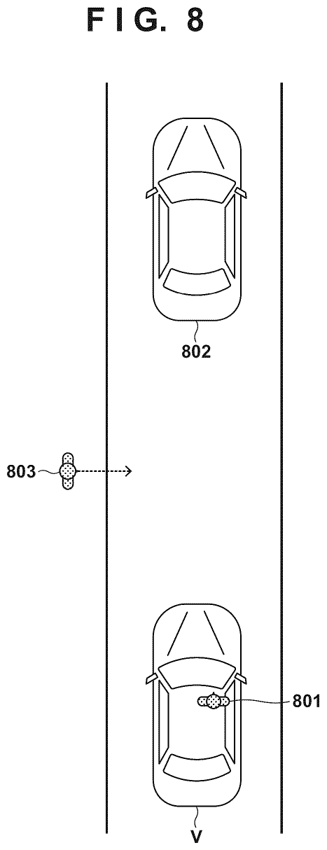

An example of a driving assistance situation will be described with reference to FIG. 8. It is assumed that a driver 801 of the vehicle Vis traveling while watching a preceding vehicle 802. The control device CNT may determine that the line of sight of the driver is directed to the front with respect to the trunk of the driver 801. In addition, the control device CNT may determine that the light environment around the vehicle Vis sunny in the daytime or that a traffic participant around the preceding vehicle 802 is only a pedestrian 803.

Furthermore, the control device CNT may measure the color of the clothes of the pedestrian 803. The control device CNT may estimate the visual field of the driver 801 based on the values of these parameters and provide driving assistance based on this visual field. The movement characteristic and the color of the target around the vehicle V can vary for each target. Therefore, the control device CNT may provide driving assistance for each traffic participant based on the visual field estimated for each target.

### Summary of Embodiments

### [Item 1]

A method for determining a visual field of a subject (300), the method comprising:
relatively moving (S202) a target (302, 305) with respect to the subject; and
determining (S205) a visual field of the subject based on a position (304, 307) of the target at a point in time when the subject has directed a line of sight of the subject to the target relatively moving with respect to the subject.

According to this item, it is possible to measure the visual field according to a situation.

### [Item 2]

The method according to Item 1, wherein
relatively moving the target includes:
   moving the target so as to approach a direction (301) of the line of sight of the subject; and
   moving the target so as to be away from the direction of the line of sight of the subject.

According to this item, it is possible to measure the visual field according to a situation in which driving assistance is provided.

### [Item 3]

The method according to Item 1 or 2, wherein relatively moving the target includes moving the subject.

According to this item, it is possible to measure the visual field according to a situation in which driving assistance is provided.

### [Item 4]

The method according to any one of Items 1-3, wherein determining the visual field of the subject includes detecting a direction of the line of sight of the subject using a measurement device (9a).

According to this item, it is possible to accurately detect the direction of the line of sight of the subject.

### [Item 5]

The method according to any one of Items 1-4, wherein determining the visual field of the subject includes determining, for each of a plurality of directions with respect to the subject, a visual field of the subject when the target is moved while the line of sight of the subject is directed in an individual direction.

According to this item, it is possible to measure the visual field in an individual situation.

### [Item 6]

The method according to any one of Items 1-5, wherein determining the visual field of the subject includes determining, for each of a plurality of targets with different shapes, a visual field of the subject when an individual target is relatively moved with respect to the subject.

According to this item, it is possible to measure the visual field in an individual situation.

### [Item 7]

The method according to any one of Items 1-6, wherein determining the visual field of the subject includes determining, for each of a plurality of movement characteristics, a visual field of the subject when the target or the subject is moved with an individual movement characteristic.

According to this item, it is possible to measure the visual field in an individual situation.

### [Item 8]

The method according to any one of Items 1-7, wherein determining the visual field of the subject includes determining, for each of a plurality of light environments, a visual field of the subject in an individual light environment.

According to this item, it is possible to measure the visual field in an individual situation.

### [Item 9]

The method according to any one of Items 1-8, wherein determining the visual field of the subject includes determining, for each of a plurality of targets with different colors, a visual field of the subject when an individual target is relatively moved with respect to the subject.

According to this item, it is possible to measure the visual field in an individual situation.

### [Item 10]

The method according to any one of Items 1-9, further comprising:
recording (S206), in a database (10), the determined visual field of the subject in association with a value of a parameter used in the determination of the visual field, wherein
the parameter includes at least one of:
   a movement characteristic of the target;
   a movement characteristic of the subject;
   a color of the target;
   a direction of the line of sight of the subject before the subject directs the line of sight to the target; and
   a light environment in which the measurement has been performed.

According to this item, it is possible to use the visual field measured in an individual situation.

### [Item 11]

A moving body (V) configured to:
estimate a visual field of a driver (801) so as to have a shape formed by a conical portion (501a) having the driver of the moving body as a vertex and a cylindrical portion (501b) extending from a bottom surface of the conical portion; and
provide driving assistance based on the estimated visual field.

According to this item, it is possible to accurately estimate the visual field to be used for driving assistance.

The invention is not limited to the foregoing embodiments, and various variations/changes are possible within the spirit of the invention.

What is claimed is:

1. A method for determining a visual field of a subject, the method comprising:
relatively moving a target with respect to the subject; and
determining a visual field of the subject based on a position of the target at a point in time when the subject has directed a line of sight of the subject to the target relatively moving with respect to the subject.

2. The method according to claim 1, wherein
relatively moving the target includes:
moving the target so as to approach a direction of the line of sight of the subject; and
moving the target so as to be away from the direction of the line of sight of the subject.

3. The method according to claim 1, wherein relatively moving the target includes moving the subject.

4. The method according to claim 1, wherein determining the visual field of the subject includes detecting a direction of the line of sight of the subject using a measurement device.

5. The method according to claim 1, wherein determining the visual field of the subject includes determining, for each of a plurality of directions with respect to the subject, a visual field of the subject when the target is moved while the line of sight of the subject is directed in an individual direction.

6. The method according to claim 1, wherein determining the visual field of the subject includes determining, for each of a plurality of targets with different shapes, a visual field of the subject when an individual target is relatively moved with respect to the subject.

7. The method according to claim 1, wherein determining the visual field of the subject includes determining, for each of a plurality of movement characteristics, a visual field of the subject when the target or the subject is moved with an individual movement characteristic.

8. The method according to claim 1, wherein determining the visual field of the subject includes determining, for each of a plurality of light environments, a visual field of the subject in an individual light environment.

9. The method according to claim 1, wherein determining the visual field of the subject includes determining, for each of a plurality of targets with different colors, a visual field of the subject when an individual target is relatively moved with respect to the subject.

10. The method according to claim 1, further comprising:
recording, in a database, the determined visual field of the subject in association with a value of a parameter used in the determination of the visual field, wherein
the parameter includes at least one of:
a movement characteristic of the target;
a movement characteristic of the subject;
a color of the target;
a direction of the line of sight of the subject before the subject directs the line of sight to the target; and
a light environment in which the measurement has been performed.

11. A moving body configured to:
estimate a visual field of a driver so as to have a shape formed by a conical portion having the driver of the moving body as a vertex and a cylindrical portion extending from a bottom surface of the conical portion; and
provide driving assistance based on the estimated visual field.
